(19) United States
(12) Patent Application Publication
(10) Pub. No.: US 2016/0051783 A1
(43) Pub. Date: Feb. 25, 2016

(54) FACEPLATE AND FACEPLATE SIZING METHOD

(71) Applicant: KONINKLIJKE PHILIPS N.V., Eindhoven (NL)

(72) Inventors: PETER CHI FAI HO, PITTSBURGH, PA (US); RICHARD ANDREW SOFRANKO, FINLEYVILLE, PA (US)

(21) Appl. No.: 14/779,606

(22) PCT Filed: Mar. 24, 2014
(86) PCT No.: PCT/IB2014/060089

§ 371 Date: Sep. 24, 2015

Related U.S. Application Data
(60) Provisional application No. 61/805,192, filed on Mar. 26, 2013.

(51) Int. Cl. A61M 16/06

(52) U.S. Cl. CPC A61M 16/0622; A61M 2016/0661; A61M 2207/00

(57) ABSTRACT

A faceplate (22) for use with a cushion (24) in a patient interface device includes a generally triangular shaped body portion (26) having a first side adapted to engage the cushion, a second side (30) disposed opposite the first side, and an aperture (32) formed therein passing between the first side and the second side. The body portion has a height (h) between a bottom edge and an apex edge and a width (a). The ratio of the width to the height is within the range of about 0.85% to 1.15% of the golden ratio (a+b/a=a/b).

### CROSS-REFERENCE TO RELATED APPLICATIONS

This patent application claims the priority benefit under 35 U.S.C. §119(e) of U.S. Provisional Application No. 61/805,192 filed on Mar. 26, 2013, the contents of which are herein incorporated by reference.

### BACKGROUND OF THE INVENTION

1. Field of the Invention

The present invention generally relates to patient interface systems for supplying a flow of gas to the airway of a patient, and, more particularly, to a method for sizing a faceplate for use in a patient interface. The invention further relates to a faceplate sized in accordance with such method, and also to a patient interface including a faceplate sized in accordance with such method.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NW). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a generally rigid or semi-rigid mask shell or faceplate having a flexible cushion attached to the faceplate that contacts, typically in a sealing fashion, the surface of the patient. The faceplate and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort.

In order to provide the best fit possible, recent approaches have been aimed at custom fitting cushions to the unique facial features and dimensions of a particular patient. However, such custom cushions can be oddly shaped and present a generally less than pleasing appearance. Such appearance is generally not helped by the use of off the shelf, one size fits all, faceplates that are typically used with such cushions.

### SUMMARY OF THE INVENTION

Accordingly, as one aspect of the present invention, a method of sizing a body portion of a faceplate for use with a cushion in forming a patient interface device is provided. The method comprises: determining a distance between a bottom portion of the cushion which is adapted to engage the face of a patient generally between the patient's lower lip and chin and an apex portion of the cushion which is adapted to engage the face of the patient at or about an upper portion of the patient's nose at or about a point generally between the patient's eyes; and forming the faceplate with the body portion having a first side adapted to engage the cushion, a second side disposed opposite the first side, and an aperture formed therein passing between the first side and the second side. The body portion has a height between a bottom edge and an apex edge which is equal to the distance between the bottom portion of the cushion and the apex portion of the cushion and a width, wherein the ratio of the width to the height is within the range of about 0.85% to 1.15% of the golden ratio. The ratio of the width to the height may be generally equal to the golden ratio.

As another aspect of the present invention, a faceplate for use with a cushion in a patient interface device is provided. The faceplate includes: a generally triangular shaped body portion having a first side adapted to engage the cushion, a second side disposed opposite the first side, and an aperture formed therein passing between the first side and the second side. The body portion has a height between a bottom edge and an apex edge and a width, wherein the ratio of the width to the height is within the range of about 0.85% to 1.15% of the golden ratio.

The ratio of the width to the height may be generally equal to the golden ratio.

The aperture may be disposed about a central point, the central point being disposed a first distance from the apex edge and a second distance from the bottom edge, wherein the ratio of the first distance to the second distance is generally equal to the golden ratio. The aperture may be of generally circular shape.

As yet a further aspect of the invention, a patient interface device is provided which comprises: a cushion having a bottom portion adapted to engage the face of a patient generally between the patient's lower lip and chin and an apex portion adapted to engage the face of the patient at or about an upper portion of the patient's nose at or about a point generally between the patient's eyes; and a faceplate comprising a generally triangular shaped body portion having a first side in engagement with the cushion, a second side disposed opposite the first side, and an aperture formed therein passing between the first side and the second side, the body portion having a height between a bottom edge and an apex edge and a width, wherein the distance between the bottom portion and the apex portion defines a cushion height, wherein the height of the body portion is equal to the cushion height and wherein the ratio of the width to the height of the body portion is within the range of about 0.85% to 1.15% of the golden ratio. The ratio of the width to the height of the body portion may be generally equal to the golden ratio.

The cushion may be custom dimensioned from one or more dimensions of facial landmarks obtained from the face of a particular patient for which the patient interface device is intended.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an example known system adapted to provide a regimen of respiratory therapy to a patient;

FIG. 2A is an isometric view showing a portion of the front and side of a patient interface device according to one exemplary embodiment of the invention;

FIG. 2B is a front elevation view of the patient interface device of FIG. 2A;

FIG. 2C is a rear (patient side) elevation view of the patient interface device of FIG. 2A;

FIG. 3A is an isometric view showing a portion of the front and side of a patient interface device according to another exemplary embodiment of the invention;

FIG. 3B is a front elevation view of the patient interface device of FIG. 3A;

FIG. 3C is a rear (patient side) elevation view of the patient interface device of FIG. 3A; and

FIG. 4 is front and rear elevation views of a variety of patient interface devices according to exemplary embodiments of the invention.

### DETAILED DESCRIPTION OF THE EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed, herein, the statement that two or more parts or components are “coupled” together shall mean that the parts are joined or operate together either directly or through one or more intermediate parts or components. As employed herein, the statement that two or more parts or components “engage” one another shall mean that the parts exert a force against one another either directly or through one or more intermediate parts or components. As employed herein, the term “number” shall mean one or an integer greater than one (i.e., a plurality) and the singular form of “a”, “an”, and “the” include plural referents unless the context clearly indicates otherwise.

As employed herein, the term “golden ratio” shall be used to refer to the ratio of two unequal quantities, wherein the ratio of the sum of the quantities to the larger quantity is equal to the ratio of the larger quantity to the smaller one. Expressed algebraically:

$$golden ratio = ϕ = \frac{( a + b )}{a} = \frac{a}{b} = 1.6180339887$$

Embodiments of the invention provide a mandate for the design of the faceplate used for masks, such as, for example, without limitation, with customized fitted masks, based on individual facial data obtained from a particular patient via direct measurement, facial scan, or other suitable method. Embodiments of the invention selectively employ the golden ratio to size the faceplate used in conjunction with a particular cushion and or a particular patient. Embodiments of the invention generally provide a “pleasing” appearance to patient interface devices in general and particularly in such devices which employ custom sized cushions which are typically oddly shaped and do not follow much proportion or similarity. By following the golden ratio, the faceplate does not need to be perfectly symmetrical in order to still maintain a coherent look among various sized cushions tailored to various individuals. Although the golden ratio is generally known and has been applied to architectural works, Applicants are unaware of the golden ratio being applied to the dimensioning of faceplates as described herein.

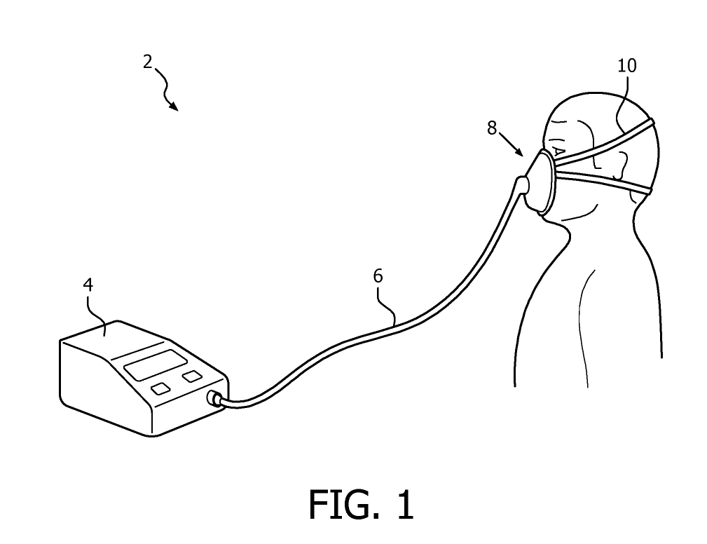

An example known system 2 adapted to provide a regimen of respiratory therapy to a patient is generally shown in FIG. 1. System 2 includes a pressure/flow generator 4, a delivery conduit circuit 6, a patient interface device 8 and a headgear 10 for securing patient interface device 8 to the head of a patient (not numbered). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8. Delivery conduit 6 and patient interface device 8 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems.

In the illustrated example system 2 of FIG. 1, patient interface device 8 is depicted as a nasal/oral mask which includes a patient sealing assembly in the form of a cushion 10 coupled to a generally rigid frame member of faceplate 12 which may be coupled to conduit 6 either directly or indirectly via any suitable coupling mechanism.

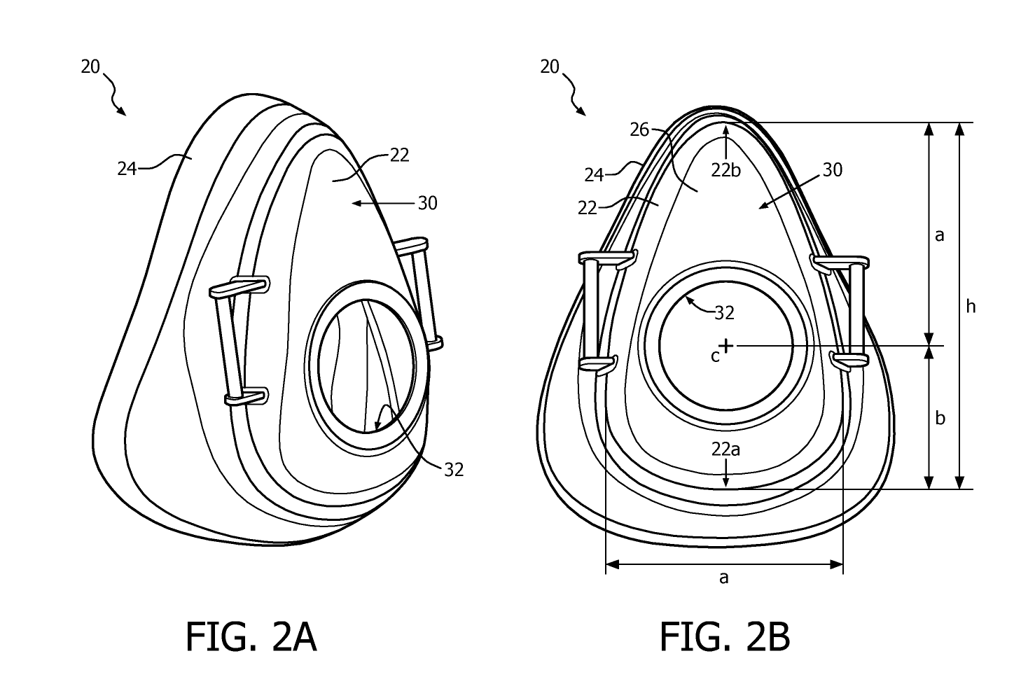

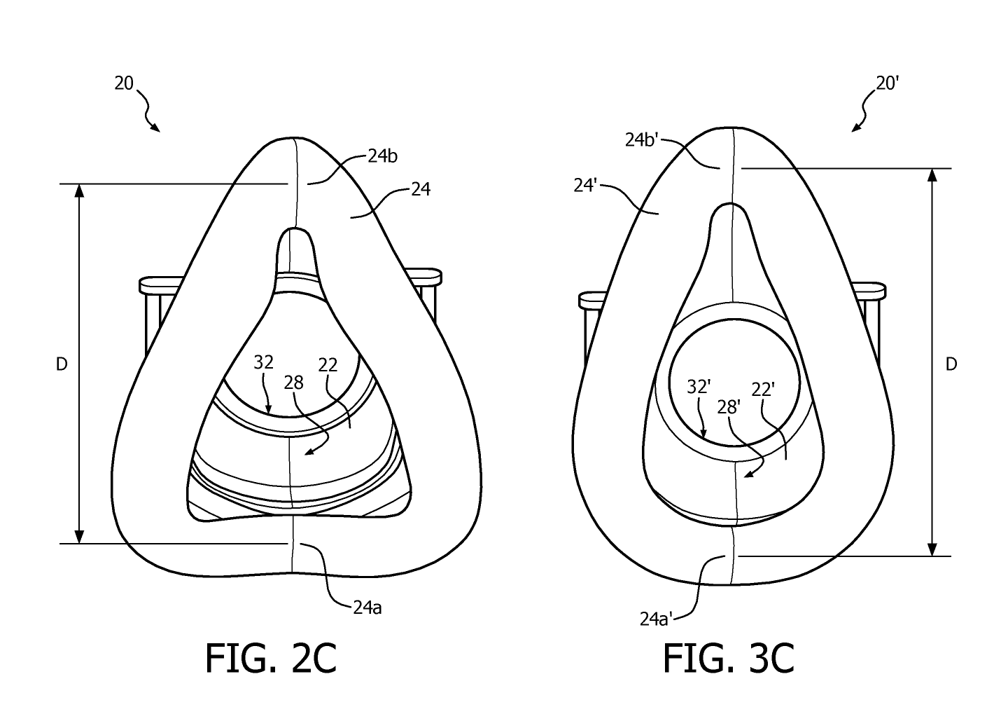

FIGS. 2A-2C illustrate a patient interface device 20 in accordance with an exemplary embodiment of the present invention. Similar to patient interface device 8 of FIG. 1, patient interface device 20 includes a generally rigid faceplate 22 and a cushion 24 for sealingly engaging the face of a patient (not shown). Cushion 24 is of generally triangular shape and is structured to be disposed about the nose and mouth of a patient. In the example illustrated embodiment, cushion 24 was formed as a custom-fit cushion based on measurements of facial landmarks taken from the particular patient to which interface device 20 is intended, it is to be appreciated, however, that cushion 24 could also be simply selected from an array of standard non-custom masks.

Measurement of facial landmarks may readily be obtained, for example, without limitation, via facial scanning, direct measurement, pin arrays, or any other technique for gathering such information, without varying from the scope of the present invention. As shown in the patient side view of FIG. 2C, cushion 24 includes a bottom portion 24a adapted to engage the face of a patient generally between a patient's lower lip and chin and an apex portion 24b adapted to engage the face of a patient at or about an upper portion of the patient's nose at or about a point generally between the patient's eyes. As shown in FIG. 2C, the distance between bottom portion 24a and apex portion 24b is denoted as the distance D.

As perhaps best shown in the elevation view of FIG. 2B, faceplate 22 has a generally triangular shaped body portion 26 having a first side 28 (FIG. 2C) adapted to engage cushion 24, a second side 30 disposed opposite first side 28, and an aperture 32 formed therein passing between first side 28 and second side 30 which is adapted to be coupled to a conduit or other connecting mechanism for receiving a flow of gas from a pressure generating device, such as pressure generating device 4 previously described in regard to FIG. 1. Similar to cushion 24 which was custom sized to a particular patient, faceplate 22 was custom dimensioned based on particular dimensions of one or both of cushion 24 and the patient.

For example, in the illustrated embodiment, body portion 26 is of a height h, as measured between a bottom edge 22a and an apex edge 22b of body portion 26. Height h is equivalent to the distance D (FIG. 2C) between a bottom portion 24a and an apex portion 24b of cushion 24. Once the height h for body portion 26 is determined, the width “a” for body portion 26 is determined using the golden ratio equation:

$$ϕ = \frac{( a + b )}{a} = \frac{h}{a} ;$$

$$hence a = \frac{h}{ϕ}$$

In addition to providing for the proportioning of body portion 26, the golden ratio is also employed to provide for the vertical positioning (height) of aperture 32 which, in the example illustrated embodiment is generally circular in shape and disposed about a center point C (FIG. 2B). As shown in FIG. 2B, center point C is spaced downward in body portion 26 an equal distance “a” from apex edge 22a as width “a”, and a distance “b” (as can be determined from the previous equation) from bottom edge 22.

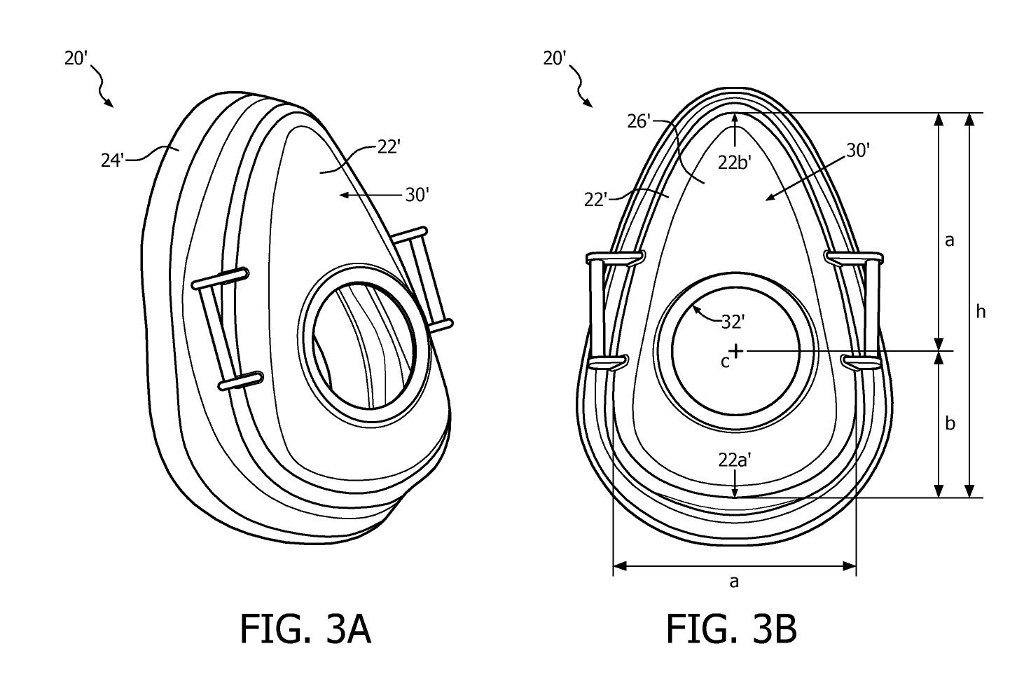

FIGS. 3A-3C illustrate another example patient interface device 20′ having a custom sized cushion 24′ and a faceplate 22′ sized in the same manner as faceplate 22 previously described. As is readily apparent from the patient side elevation views of FIGS. 2C and 3C, cushions 24 and 24′ are of vastly different shapes. However, by employing the faceplate sizing method described herein, the two patient interface devices 20 and 20′ appear quite similar and aesthetically pleasing when viewed from the front sides, such as shown in FIGS. 2B and 3B.

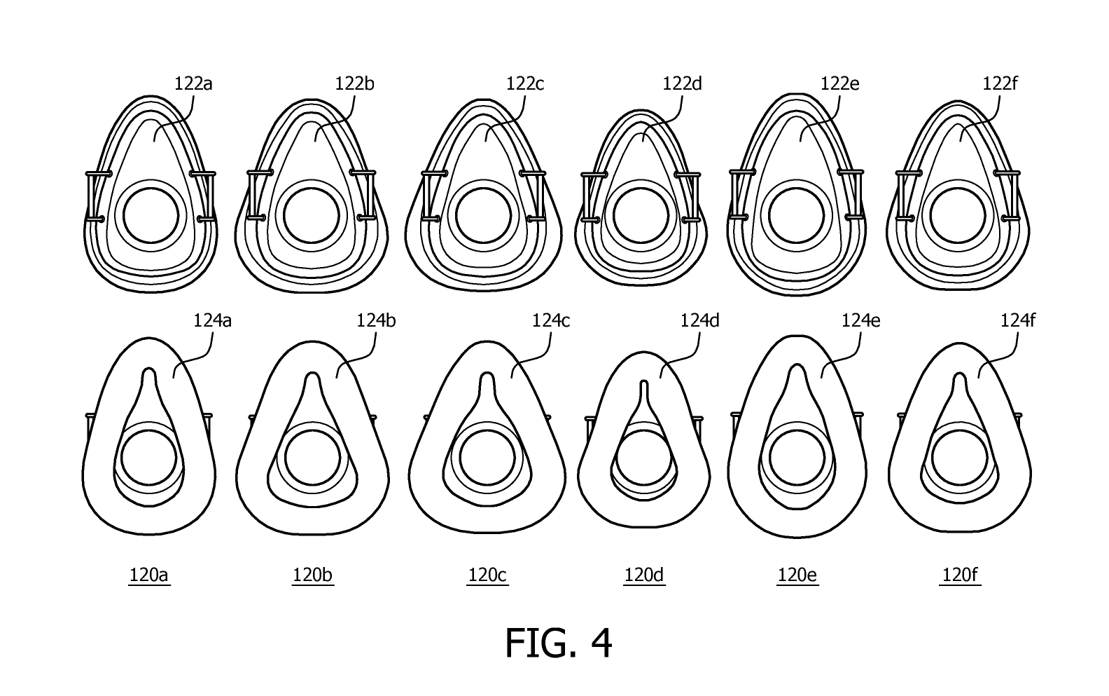

FIG. 4 further illustrates benefits of the invention by showing front and rear (patient side) elevation views of a plurality of patient interface devices 20a-20f, each having a custom sized cushion 24a-24f and a faceplate 22a-22f sized in the same manner as faceplate 22 as previously described. Once again, it can be readily appreciated that although cushions 24a-24f are of different sizes and shapes, by using the method described herein to size each of faceplates 22a-22f, each of patient interface devices 20a-20f share a common related appearance that is generally aesthetically pleasing.

It can be appreciated that the present invention provides a method for sizing faceplates that improves upon existing methods, for example, to provide faceplates sized for customized cushions of various sizes which improves and provides a degree of uniformity to the appearance of such cushions.

While the example embodiments described herein include faceplates having height to width ratios generally equal to the golden ratio, it is to be appreciated that due to variations in manufacturing, actual faceplate dimensions which produce a ratio within 15% (plus or minus) of the golden ratio φ (i.e., about 0.85φ-1.15φ) have been found to generally produce desirable results in accordance with the present invention. However, ratios as close to the golden ratio are preferred.

Although the invention has been described in detail for the purpose of illustration based on what is currently considered to be the most practical example embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A faceplate for use with a cushion in a patient interface device, the faceplate comprising:
a generally triangular shaped body portion having a first side adapted to engage the cushion, a second side disposed opposite the first side, and an aperture formed therein passing between the first side and the second side, the body portion having a height between a bottom edge and an apex edge and a width, wherein the ratio of the width to the height is within the range of about 0.85% to 1.15% of the golden ratio (φ).

2. The faceplate of claim 1, wherein the ratio of the width to the height is generally equal to the golden ratio (φ).

3. The faceplate of claim 1, wherein the aperture is disposed about a central point, the central point being disposed a first distance from the apex edge and a second distance from the bottom edge, and wherein the ratio of the first distance to the second distance is generally equal to the golden ratio.

4. The faceplate of claim 1, wherein the aperture is of generally circular shape.

5. A patient interface device comprising:
a cushion having a bottom portion adapted to engage the face of a patient generally between the patient's lower lip and chin and an apex portion adapted to engage the face of the patient at or about an upper portion of the patient's nose at or about a point generally between the patient's eyes; and
a faceplate comprising a generally triangular shaped body portion having a first side in engagement with the cushion, a second side disposed opposite the first side, and an aperture formed therein passing between the first side and the second side, the body portion having a height between a bottom edge and an apex edge and a width, wherein the distance between the bottom portion and the apex portion defines a cushion height, wherein the height of the body portion is equal to the cushion height and wherein the ratio of the width to the height of the body portion is within the range of about 0.85% to 1.15% of the golden ratio (φ).

6. The patient interface device of claim 5, wherein the ratio of the width to the height of the body portion is generally equal to the golden ratio (φ).

7. The patient interface device of claim 5, wherein the aperture of the faceplate is disposed about a central point which is disposed a first distance from the apex edge and a second distance from the bottom edge, and wherein the ratio of the first distance to the second distance is generally equal to the golden ratio (φ).

8. The patient interface device of claim 5, wherein the aperture of the faceplate is of generally circular shape.

9. The patient interface device of claim 5, wherein the cushion is custom dimensioned from one or more dimensions of facial landmarks obtained from the face of a particular patient for which the patient interface device is intended.

10. A method of sizing a body portion of a faceplate for use with a cushion in forming a patient interface device, the method comprising:
determining a distance between a bottom portion of the cushion which is adapted to engage the face of a patient generally between the patient's lower lip and chin and an apex portion of the cushion which is adapted to engage the face of the patient at or about an upper portion of the patient's nose at or about a point generally between the patient's eyes; and
forming the faceplate with the body portion having a first side adapted to engage the cushion, a second side disposed opposite the first side, and an aperture formed therein passing between the first side and the second side, the body portion having a height between a bottom edge and an apex edge which is equal to the distance and a width (a), wherein the ratio of the width to the height is within the range of about 0.85% to 1.15% of the golden ratio (φ).

11. The method of claim 10, wherein the ratio of the width to the height is generally equal to the golden ratio (φ).
